(19) United States
(12) Patent Application Publication
(10) Pub. No.: US 2015/0110903 A1
(43) Pub. Date: Apr. 23, 2015

(54) METHOD AND PRODUCT FOR HEADACHE RELIEF

(71) Applicant: AMBERWING SOLUTIONS INC., ROCKFORD, IL (US)

(72) Inventors: Mohsin A. Khan, Rockford, IL (US); Liliana Brotea, West Caldwell, NJ (US); Melwyn A. Abreo, Jamul, CA (US)

(73) Assignee: AMBERWING SOLUTIONS INC., ROCKFORD, IL (US)

(21) Appl. No.: 14/398,459

(22) PCT Filed: May 1, 2013
(86) PCT No.: PCT/US13/00126

§ 371 Date: Nov. 1, 2014

Related U.S. Application Data
(60) Provisional application No. 61/640,961, filed on May 1, 2012.
(60) Provisional application No. 61/801,874, filed on Mar. 15, 2013.

(51) Int. Cl. A61K 36/906; A61K 36/534; A61K 31/045; A61K 31/355; A61K 45/06; A61K 31/353; A61K 31/522; A61K 36/537; A61K 31/4166; A61K 36/258; A61K 36/82

(52) U.S. Cl. CPC A61K 36/906; A61K 36/258; A61K 36/534; A61K 31/045; A61K 31/355; A61K 36/82; A61K 31/353; A61K 31/522; A61K 36/537; A61K 31/4166; A61K 45/06
USPC 424/728; 424/747

(57) ABSTRACT

A novel product for headache relief and use of the product to treat headaches is disclosed herein. The product may be applied topically, and may be used for acute treatment of headaches to provide a safe, effective, and natural alternative to traditional therapies used to treat acute headache syndromes. The product comprises one or more herbal therapeutic agents. In some embodiments, the one or more herbal therapeutic agents may be combined with allopathic or other traditional headache therapies. In preferred embodiments, the product is applied to select myofascial trigger points. (MTPs) on the temporal, frontal, mastoid, and cervical regions. The product may be available over-the-counter or otherwise may be available without a physician's prescription.

### CROSS-REFERENCE TO RELATED APPLICATIONS

This application claims priority to U.S. Provisional Patent Appl. Nos. 61/640,961 and 61/801,874, filed on May 1, 2012 and Mar. 15, 2013 respectively, the disclosures of which are incorporated herein in their entireties by reference.

### BACKGROUND

1. Field of the Invention

The present disclosure describes a novel product for headache relief and use of the product to treat headaches.

2. Description of the Related Art

Headaches are among the most common medical complaints and may lead to significant discomfort and disability.¹ According to the World Health Organization, headaches affect approximately ⅔ of men and over 80% of women worldwide.² One in twenty adults suffers from one or more headaches every day or nearly every day.2 A survey in 1993 demonstrated that 150 million lost workdays and 329,000 lost school days each year in the United States are attributable to headaches.^{3,4} Most benign headaches fall into one of three categories: tension headaches, migraine headaches, and cluster headaches.1 While a population-based study found a 38 percent one-year prevalence of episodic tension-type headaches,⁵ most individuals do not seek medical treatment from physicians for headaches.1 While the International Headache Society (HIS) has classified primary headaches into the aforementioned syndromes⁶ for the purpose of study, many clinicians appreciate the overlap between the categories and employ a mechanism-based approach to diagnosis and management of headaches. ¹http://www.uptodate.com²http://www.who.int/features/qa/25/en/index.html³Stang, P. E.; Osterhaus, J. T. “Impact of Migraine in the United States: Data from the National Health Interview Survey.” Headache 1993, 33:29.⁴Stewart, W. F.; Lipton, R. B.; Celentano, D. D.; Reed, M. L. “Prevalence of Migraine Headache in the United States: Relation to Age, Income, Race, and Other Sociodemographic Factors.” J. Am. Med. Assoc. 1992, 267:64.⁵http://www.aafp.org/afp/2007/0401/p1027.html⁶Headache Classification Committee of the International Headache Society. “Classification and Diagnostic Criteria for Headache Disorders, Cranial Neuralgias and Facial Pain.” Cephalalgia 1988, 8 Suppl. 7:1.

Despite extensive research, the exact mechanism of primary headaches remains unclear. Rather, several pathophysiological pathways have been identified as contributors to head pain and associated headache symptoms. These pathways include muscular tension,^{7,8} cortical spreading depression,1 central sensitization,8 neurogenic inflammation,⁹ and activation of the trigeminovascular system.1 ⁷Lyngberg, A. C.; Rasmussen, B. K.; Jørgensen, T.; Jensen, R. “Incidence of Primary Headache: A Danish Epidemiologic Follow-Up Study.” Am. J. Epidemiol. 2005, 161:1066.⁸Fernández-de-las-Peñas, C.; Cuadrado, M. L.; Arendt-Nielsen, L.; Simons, D. G.; Pareja, J. A. “Myofascial Trigger Points and Sensitization: An Updated Pain Model for Tension-Type Headache.” Cephalalgia 2007, 27(5):383.⁹Fusco, M.; D'Andrea, G.; Miccichè, F.; Stecca, A.; Bernardini, D.; Cananzi, A. L. “Neurogenic inflammation in primary headaches.” Neurol. Sci. 2003, 24 Suppl. 2:S61.

Myofascial trigger points (MTPs) play a facilitating role in most common headache types, including migraine, tension, neuralgic, and cluster headaches. MTPs have been known to practitioners of traditional medicine for centuries. In addition, the pathophysiology of MTPs has recently been studied extensively and the emerging data is helping to outline the role of MTPs in headache causation. However, the exact physiology of MTPs in facilitating headaches is currently not fully understood.8^{,10} MTPs are present over distinct cranial nerve distributions. The important nerve distributions involved in headaches are shown in FIG. 1, and the MTPs associated with these distributions are shown in FIG. 2. The MTPs are associated with referred pain, as shown in FIG. 3. Referred pain from MTPs in the cervical, suprascapular, and occipital regions contributes to central sensitization of the trigeminal nerve nucleus. Muscle pain is produced mainly by noxious stimuli that lead to increased synthesis and release of endogenous allogeneic substances such as serotonin, bradykinin, histamine, and prostaglandins. Table 1 provides details regarding the location of selected MTPs, the nerve distributions and muscles related to said MTPs, and the areas to which pain is referred from those MTPs. ¹⁰Calandre, E. P.; Hidalgo, J.; Garcia-Leiva, J. M.; Rico-Villademoros, F.; Delgado-Rodriguez, A. “Myofascial Trigger Points in Cluster Headache Patients: A Case Series.” Head Face Med. 2008, 4:32.

TABLE 1

| Trigger | | | | | |
|---|---|---|---|---|---|
| Point | Side | Region | Nerve Distribution | Muscles Involved | Pain Referred To |
| M1 | LEFT | Mastoid | Lesser Occipital | Left Suboccipitalis | Left Frontal |
| | | | Greater Auricular | Left Sternocleidomastoid | Temporal |
| | | | | | Apex |
| | | | | | Occipital |
| | | | | | Cervical |
| M2 | RIGHT | Mastoid | Lesser Occipital | Right Suboccipitalis | Right Frontal |
| | | | Greater Auricular | Right Sternocleidomastoid | Temporal |
| | | | | | Apex |
| | | | | | Occipital |
| | | | | | Cervical |
| F1 | LEFT | Frontal | Supraorbital | Left Frontalis | Left Frontal |
| | | | Supratrochlear | | Apex |
| F2 | RIGHT | Frontal | Supraorbital | Right Frontalis | Right Frontal |
| | | | Supratrochlear | | Apex |
| T1 | LEFT | Temporal | Auriculotemporal | Left Temporalis | Left Maxillary |
| | | | | | Frontal |
| | | | | | Temporal |
| | | | | | Occipital |
| | | | | | Cervical |
| T2 | RIGHT | Temporal | Auriculotemporal | Right Temporalis | Right Maxillary |
| | | | | | Frontal |
| | | | | | Temporal |
| | | | | | Occipital |
| | | | | | Cervical |
| C1 | LEFT | Mid-Cervical | C2-C4 | Left Cervical Trapezius | Left Occipital |
| | | | | Splenius Capitus | Apex |
| | | | | Splenius Cervicis | Frontal |
| | | | | Semispinalis Cervicis | |
| C2 | RIGHT | Mid-Cervical | C2-C4 | Right Cervical Trapezius | Right Occipital |
| | | | | Splenius Capitus | Apex |
| | | | | Splenius Cervicis | Frontal |
| | | | | Semispinalis Cervicis | |

The cervical roots c1, c2, and c3 and trigeminal branches 1 and 3 converge on the same set of neurons in the trigeminal nerve nucleus and thus pain generated in the neck, shoulders, and occiput is referred to the head and trigeminal distribution. Additionally, pain generated in the anterior and posterior temporalis muscles is referred to the trigeminal nerve distribution and cervical dermatomes, and thus myofascial pain in the temporal MTPs leads to facial, head, and neck pain. FIG. 3A shows how pain radiates from the cervical trigger points in the trapezius (1), semispinalis capitus (2), and splenius capitus and splenius cervicis (3) muscles. FIG. 3B shows how pain radiates from the trigger points in the frontalis (4) muscles. FIG. 3C shows how pain radiates from trigger points in the occipitalis (5) and sternocleidomastoid (6) muscles. FIG. 3D shows how pain radiates from trigger points in the temporalis (7) muscles.

The release of MTPs leads to improvement in underlying headaches. MTPs may be released mechanically, chemically, or surgically. Mechanical release of MTPs through various forms of massage therapy may provide effective acute relief of headaches as well as improve mood.¹¹ Chemical release of MTPs may be achieved through pharmacotherapy, such as botulinum toxin therapy. Surgical release of MTPs is currently being studied, with promising results.¹² ¹¹Moraska, A.; Chandler, C. “Changes in Psychological Parameters in Patients with Tension-Type Headache Following Massage Therapy: A Pilot Study.” J. Man. Manip. Ther. 2009, 17(2):86.¹²http://www.sciencedaily.com/releases/2007/02/070227105323.htm

Current abortive therapies for management of common headache syndromes include non-steroidal anti-inflammatory drugs (NSAIDs) such as ibuprofen and naproxen, non-opiod analgesics such as acetaminophen, and tryptans such as sumatriptan which reduce vascular dilation and vasogenic-mediated pain. In addition, antiemetics such as ondansetron and metoclopramide are used to reduce nausea that often accompanies headaches. The aforementioned treatments are generally effective, but are limited due to side effects, analgesic overuse, and rebound headache phenomena. Furthermore, these therapies are of limited use in situations when a non-oral treatment route is most appropriate. Currently available natural, non-oral headache treatments are primarily focused on peppermint oil or menthol and aromatherapy, and are not specifically targeted toward release of MTPs or other specific physiological targets for treatment. In addition to the relative dearth of safe, natural alternatives to standard oral headache therapies, most currently available alternatives do not provide specific physiological targets to focus treatment. The MIGRASTICK topically delivers essential oils including peppermint oil and lavender oil for headache treatment.¹³ Lavender oil is known to induce migraine headaches in certain individuals with smell sensitivity,¹⁴ and thus headache treatment with a product comprising lavender oil may be counterproductive in certain cases. ¹³http://www.migrastick.com/en/index.php¹⁴http://www.umm.edu/altmed/articles/lavender-000260.htm

Thus there remains a need for a safe, effective, and natural alternative to traditional headache therapies which may be topically delivered to select MTPs on the temporal, frontal, mastoid, and cervical regions.

### SUMMARY

A novel product for headache relief and use of the product to treat headaches is disclosed herein. The product may be applied topically, and may be used for acute treatment of headaches to provide a safe, effective, and natural alternative to traditional therapies used to treat acute headache syndromes. The product comprises one or more herbal therapeutics. In some embodiments, the herbal therapeutic(s) may be combined with allopathic or other traditional headache therapies. In preferred embodiments, the product is applied to select myofascial trigger points (MTPs) on the temporal, frontal, mastoid, and cervical regions. The product may be available over-the-counter or otherwise may be available without a physician's prescription.

### BRIEF DESCRIPTION OF THE DRAWINGS

FIG. 1 illustrates nerve distributions that may be targeted by application of the disclosed product to one or more myofascial trigger points.

FIG. 2 illustrates the myofascial trigger points that may be targeted for application of the disclosed product.

FIG. 3 illustrates myofascial trigger points and the direction of referred pain from each trigger point.

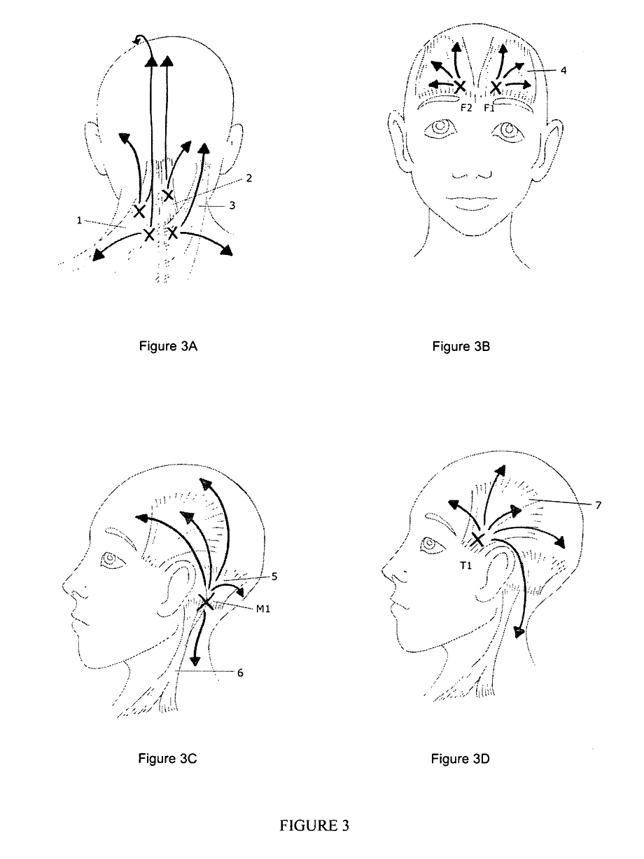

FIG. 4 illustrates bottles that may be used to deliver the disclosed product.

### DETAILED DESCRIPTION OF THE PREFERRED EMBODIMENTS

The present disclosure describes a novel product for headache relief. The product may be applied topically, and may be used for acute treatment of headaches to provide a safe, effective, and natural alternative to traditional therapies used to treat acute headache syndromes. The product comprises one or more herbal therapeutic agents. In some embodiments, the one or more herbal therapeutic agents may be combined with allopathic or other traditional headache therapies. In preferred embodiments, the product is applied to select myofascial trigger points (MTPs) on the temporal, frontal, mastoid, and cervical regions. The product may be available over-the-counter or otherwise may be available without a physician's prescription.

All herbal and non-herbal therapeutic agents are used in amounts that are not harmful and are in accordance with applicable regulations.

### Herbal Components

The product comprises one or more herbal therapeutic agents. The term herbal therapeutic agents as used herein refers to products of natural origin—i.e., non-synthetic products—that provide one or more therapeutic effects in humans and includes herbal and other non-herbal natural products that provide one or more therapeutic effects in humans. The product may comprise any suitable herbal therapeutic agents that provide one or more of the desired therapeutic effects. The herbal therapeutic agents may be selected from the group consisting of herbal or other non-herbal natural products listed in one or more of the over-the-counter (OTC) nonprescription drug product monographs established by the United States Food and Drug Administration (FDA) and other herbal or other non-herbal natural products not monographed by the FDA that have one or more therapeutic properties.

In preferred embodiments, the product comprises one or more herbal therapeutic agents selected from the group consisting of peppermint oil, menthol, ginger oil, vitamin E, green tea extract, catechin, Korean ginseng, American ginseng, caffeine, β-endorphin receptor agonists, valerian, passionflower, clary sage, rosemary, black seed oil, camphor, clove oil, capsaicin, methyl salicylate, garlic oil, lemon oil, tea tree oil, castor oil, eucalyptus oil, jojoba oil, flaxseed oil, lanolin, cumin oil, and tansy oil.

Peppermint oil and its key component compound menthol have several known therapeutic properties. Peppermint oil has analgesic,¹⁵ spasmolytic,¹⁶ anti-inflammatory,¹⁷ antiemetic,¹⁸ antioxidant,^{19,20} antimicrobial,^{20,21} and cooling²² properties. Menthol has been studied for use in abortive therapy of migraines.²³ When applied topically to the forehead and temples, menthol provides symptomic relief of headaches.²³

Ginger oil has anti-inflammatory,²⁴ analgesic,^{25,26} and warming^{27,28} properties. Vitamin E is a known antioxidant²⁹ and has anti-inflammatory properties.³⁰ Green tea extract promotes alertness,³¹ wellness,³² and euphoria.³³ Catechin is a known antioxidant.³⁴ Korean ginseng promotes alertness and wellness.³⁵ American ginseng improves focus and concentration.³⁶ Caffeine promotes alertness.³⁷ β-endorphin receptor agonists are compounds that bind to β-endorphin receptors and thereby provide analgesic effects³⁸ and promote euphoria and a sense of well-being.³⁹ Valerian promotes euphoria and clarity.⁴⁰ Passionflower promotes euphoria.⁴⁰ Clary sage promotes euphoria and inhibits migraines.⁴⁰ Rosemary promotes euphoria⁴⁰ and has sedating⁴¹ properties. Black seed oil—the oil derived from seeds of the plant Nigella sativa—has analgesic, anti-inflammatory, and numerous other therapeutic properties.⁴² Camphor has analgesic⁴³ and cooling⁴⁴ properties. Clove oil has analgesic^{45,46} and cooling⁴⁶ properties. Capsaicin has analgesic⁴⁷ and warming⁴⁸ properties. Methyl salicylate has counterirritant, anti-inflammatory, and analgesic properties.⁴⁹ ²⁴Minghetti, P.; Sosa, S.; Cilurzo, F.; Casiraghi, A.; Alberti, E.; Tubaro, A.; Loggia, R. D.; Montanari, L. “Evaluation of the Topical Anti-Inflammatory Activity of Ginger Dry Extracts from Solutions and Plasters.” Planta Med. 2007, 73(15):1525.²⁵http://www.essentialoils.co.za/essential-oils/ginger.htm²⁶Sepahvand, R.; Esmaeili-Mahani, S.; Arzi, A.; Rasoulian, B.; Abbasnejad, M. “Ginger (Zingiber Officinale Roscoe) Elicits Antinociceptive Properties and Potentiates Morphine-Induced Analgesia in the Rat Radiant Heat Tail-Flick Test.” J. Med. Food 2010, 13(6):1397.²⁷http://www.thenaturalife.com/herbalist/Article%3A_Stay_warm_and_healthy_with_ginger.html²⁸Zhao, Y. L.; Wang, J. B.; Xiao, X. H.; Zhao, H. P.; Zhou, C. P.; Zhang, X. R.; Ren, Y. S.; Jia, L. “Study on the Cold and Hot Properties of Medicinal Herbs by Thermotropism in Mice Behavior.” J. Ethnopharmacol. 2011, 133(3):980.²⁹http://vitamineoiluses.com/vitamin-e-oil-for-skin-application-benefits/³⁰http://www.md-health.com/vitamin-E-for-skin.html³¹http://www.worldhealth.net/news/green-tea-extract-boots-alertness-and-enhances-mem/³²http://www.drlarrydirect.com/pages/4-benefits-of-green-tea-for-weight-loss-and-overall-wellness/.html³³http://www.wtea.com/about-tea_health-benefits.aspx³⁴Tournaire, C.; Croux, S.; Maurette, M.-T.; Beck, I.; Hocquaux, M.; Braun, A. M.; Oliveros, E. “Antioxidant Activity of Flavonoids: Efficiency of Singlet Oxygen (¹Δ_{g}) Quenching.” J. Photochem. Photobiol. B: Biol. 1993, 19(3):205.³⁵http://www.alive.com/articles/view/17553/ginseng_for_energy_and_longevity³⁶http://www.clarocet.com/american-ginseng/³⁷Jacobson, B. H.; Kulling, F. A. “Health and Erogenic Effects of Caffeine.” Br. J. Sports Med. 2013, 23(1):34-40, available at http://bjsportmed.com/content/23/1/34.full.pdf+html³⁸Manzanares, J.; Julian, M. D.; Carrascosa, A. “Role of the Cannabinoid System in Pain Control and Therapeutic Implications for the Management of Acute and Chronic Pain Episodes.” Curr. Neuropharmacol. 2006, 4(3):239-57, available at http://www.ncbi.nlm.nih.gov/pmc/articles/PMC2430692/³⁹http://www.mibellebiochemistry.com/pdfs/Role_of_beta-endorphin_in_the_skin_SoeFW_{—}2005.pdf⁴⁰http://www.livestrong.com/article/401997-herbs-that-cause-euphoria/⁴¹http://www.oilsandplants.com/rosemary.htm⁴² http://www.theblessedseed.com/?research,29⁴³Xu, H.; Blair, N. T.; Clapham, D. E. “Camphor Activates and Strongly Desensitizes the Transient Receptor Potential Vanilloid Subtype 1 Channel in a Vanilloid-Independent Mechanism.” J. Neurosci. 2005, 25(39):8924.⁴⁴ http://en.wikipedia.org/wiki/camphor#medicinal⁴⁵ http://www.marinwater.org/documents/Chap6_CloveOil_{—}8_{—}28_{—}08.pdf⁴⁶http://healthoracle.org/downloads/C/Clove%20oil.pdf⁴⁷Dray A. “Mechanism of Action of Capsaicin-Like Molecules on Sensory Neurons.” Life Sci. 1992 51(23):1759.⁴⁸http://www.edinfonnatics.com/math_science/science_of_cooking/capsaicin.htm⁴⁹Collins, A. J.; Notarianni, L. J.; Ring, E. F. J.; Seed, M. P. “Some Observations on the Pharmacology of ‘Deep-Heat’, a Topical Rubifacient.” Ann. Rheum. Dis. 1984, 43:411.

In preferred embodiments, the product comprises one or more components selected from the group consisting of peppermint oil and menthol, a pharmaceutically acceptable carrier base, and one or more components selected from the group consisting of ginger oil, vitamin E, green tea extract, catechin, Korean ginseng, American ginseng, caffeine, β-endorphin receptor agonists, valerian, passionflower, clary sage, rosemary, black seed oil, camphor, clove oil, capsaicin, methyl salicylate, garlic oil, lemon oil, tea tree oil, castor oil, eucalyptus oil, jojoba oil, flaxseed oil, lanolin, cumin oil, and tansy oil. In more preferred embodiments, the product comprises one or more components selected from the group consisting of peppermint oil and menthol, a pharmaceutically acceptable carrier base, and one or more components selected from the group consisting of ginger oil, vitamin E, green tea extract, catechin, Korean ginseng, American ginseng, caffeine, β-endorphin receptor agonists, valerian, passionflower, clary sage, rosemary, and black seed oil.

Peppermint oil, menthol, and combinations thereof may provide one or more therapeutic effects, including analgesic, spasmolytic, anti-inflammatory, antiemetic, and cooling effects.

Ginger oil may enhance the analgesic effects of the product. Ginger oil may further provide warming effects, thereby producing a combination warming/cooling sensation that may further relieve pain.

Black seed oil may enhance the analgesic effects of the product, and may possibly provide prophylactic effects as well.

Camphor may provide one or more therapeutic effects, including analgesic and cooling effects, and may facilitate the analgesic and cooling effects of peppermint oil, menthol, or the combination thereof and thereby allow for use of a lower concentration of peppermint oil, menthol, or the combination thereof. Use of a lower concentration of peppermint oil, menthol, or a combination thereof will reduce the product's mint odor.

Clove oil may provide one or more therapeutic effects, including analgesic and cooling effects, and may facilitate the analgesic and cooling effects of peppermint oil, menthol, or the combination thereof and thereby allow for use of a lower concentration of peppermint oil, menthol, or the combination thereof.

### Carrier Base

In some embodiments, the carrier base may comprise beeswax or an equivalent wax base. In other embodiments, the carrier base may comprise a pharmaceutically acceptable liquid carrier base such as water, one or more glycols, one or more carrier oils, or a combination thereof. In some preferred embodiments, the pharmaceutically acceptable liquid carrier base may comprise water and one or more alcohols, diols, glycols, or combinations thereof. In some highly preferred embodiments, the alcohols, diols, glycols, or combinations thereof may comprise butylene glycol.

In preferred embodiments, the carrier base may provide the product with serum properties such that the product is highly absorbent, generates minimal residue, and is compatible with makeup products.

In preferred embodiments, the carrier base may comprise one or more dermal penetration enhancers. The dermal penetration enhancer may be a naturally occurring substance, including but not limited to castor oil, vegetable glycerin, laurin, jojoba oil, lemon oil, wheat germ oil, anise oil, 1-carvanone, cardamom oil, cod liver oil, eucalyptol, eugenol, menthol, nerolidol, alpha-tocopherol, ylang-ylang oil, flaxseed oil, and select terpenes. The dermal penetration enhancer may alternatively be synthetically derived, including but not limited to fatty alcohols such as lauryl alcohol, n-octanol, and oleyl alcohol; fatty acid esters such as butyl acetate, cetyl lactate, decyl N,N-dimethylamino isopropionate, diethyl sebacate, glycerol monoethers, isopropyl isostearate, isopropyl palmitate, isopropyl myristate, methyl caprate, oleyl oleate, sorbitan dilaurate, and sucrose monolaurate; fatty acids such as capric acid, lactic acid, lauric acid, linoelaidic acid, linoleic acid, linolenic acid, oleic acid, palmitic acid, and vaccenic acid; fatty alcohol ethers such as α-monoglyceryl ether and ether derivatives of polyglycerols and alcohols; biologics such as lecithin, phospholipids, saponin, sodium deoxycholate, sodium taurocholate, and sodium tauroglycocholate; enzymes such as acid phosphonate, calonase, orgelase, papain, phospholipase A-2, phospholipase C, and triacylglycerol hydrolase; amines and amides such as acetamide derivatives, acyclic amides, N-adamantyl n-alkanamides, clofibric acid amides, N,N-didodecyl acetamide, N,N-diethyl-m-toluamide, hexamethylene lauramide, dodecylamine, octyl amide, oleylamine, unsaturated cyclic ureas, and urea; complexing agents such as β- and γ-cyclodextrin complexes, hydroxypropyl methylcellulose, liposomes, ethylenediaminetetraacetate (EDTA) salts, and naphthalene diamide diimide; macrocyclics such as macrocyclic lactones, ketones and anhydrides, and unsaturated cyclic ureas; surfactants such as polysorbates, cetyl trimethyl ammonium bromide, hydroxypolethoxydodecane, ionic surfactants, decyl glucoside, lauroyl sarcosine, nonionic surface active agents, nonoxynol, pluronic F68, pluronic F127, pluronic L62, polyoleates, sodium laurate, sodium lauryl sulfate, sodium oleate, sodium dilaurate, sorbitan dioleate, sorbitan monolaurate, span 20, span 40, triton X-100, tween-20, -40, -60, -80, and -85; N-methyl pyrrolidones such as N-cyclohexyl-2-pyrrolidone, N-methyl-2-pyrrolidone, poly(N-vinylpyrrolidone), and 2-pyrrolidone; ionic compounds such as ascorbate, amphoteric cations and anions, calcium thioglycolate, cetyl trimethyl ammonium bromide, and 3,5-diiodosalicylate sodium; dimethyl sulfoxides such as cyclic sulfoxides, decylmethyl sulfoxides, dimethyl sulfoxide, and 2-hydroxyundecyl methyl sulfoxide; and azones and related compounds such as N-acyl-hexahydro-2-oxo-1H-azepines, N-alkylmorpholine-2,3-diones, azacycloalkane derivatives, and N-(2,2-dihydroxyethyl)dodecylamine.

In more preferred embodiments the dermal penetration enhancers may comprise one or more components selected from the group consisting of polysorbates, decyl glucoside, and ethylenediaminetetraacetate (EDTA) salts. In highly preferred embodiments the polysorbates may comprise polysorbate 20 and the ethylenediaminetetraacetate salts may comprise disodium ethylenediaminetetraacetate.

### Non-Herbal Components

In some embodiments, the one or more herbal therapeutic agents may be combined with one or more non-herbal therapeutic agents used in allopathic or other traditional headache therapies. The term non-herbal therapeutic agents as used herein refers to products of synthetic origin that provide one or more therapeutic effects in humans. The non-herbal therapeutic agents may comprise one or more agents such as non-steroidal anti-inflammatory drugs (NSAIDs), steroidal anti-inflammatory agents, analgesics, or anesthetics, or any combinations thereof.

In some embodiments, the one or more herbal therapeutic agents may be combined with one or more NSAIDs such as diclofenac, ibuprofen, ketoprofen, piroxicam, indomethacin, benzydamine, and the pro-drugs and salts of these agents; one or more steroidal anti-inflammatory drugs such as hydrocortisone, hydrocortisone acetate, cortisone acetate, tixocortol pivalate, prednisolone, methyprednisolone, prednisone, triamcinolone acetonide, triamcinolone alcohol, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, and the pro-drugs and salts of these agents; or any combinations thereof.

In some embodiments, the one or more herbal therapeutic agents may be combined with one or more allopathic agents selected from the FDA over-the-counter (OTC) monograph listed as “analgesics and anesthetics” or “external analgesics” such as aspirin, benzyl alcohol, camphorated metacresol, chloral hydrate, chlorobutanol, cyclomethycaine sulfate, eugenol, hexylresorcinol, methapyrilene hydrochloride, salicylamide, tetracaine, and thymol.

In some embodiments, the one or more herbal therapeutic agents may be combined with one or more allopathic agents selected from the FDA over-the-counter (OTC) monograph listed as “topical analgesics” such as aluminum hydroxide.

In some embodiments, the one or more herbal therapeutic agents may be combined with one or more allopathic agents selected from the FDA over-the-counter (OTC) monograph listed as “external analgesics” such as allantoin, aluminum acetate, aluminum chloride hexahydrate, benzocaine, butamben picrate, camphor, cupric sulfate, dexpanthenol, dibucaine, dibucaine hydrochloride, dimethisoquin hydrochloride, diphenhydramine hydrochloride, dyclonine hydrochloride, hydrocortisone, hydrocortisone acetate, juniper tar, lidocaine, lidocaine hydrochloride, menthol, obtundia surgical dressing, phenol, phenolate sodium, pramoxine hydrochloride, resorcinol, sodium bicarbonate, tetracaine hydrochloride, topical starch, tripelennamine hydrochloride, trolamine, zinc acetate, and zinc oxide.

In some embodiments, the one or more herbal therapeutic agents may be combined with one or more allopathic prescription-based pain medications which have the potential to modulate pathways and receptors like the COX-2 and enkephalinase enzymes, the P2X3, P2X4, P2X7, P2Y1, P2Y2, TRPV1, peripheral kappa opioid, nicotinic, NaV1.7, and mGluR receptors, and the TREK1 and TRESK channels via dermal delivery.

### Combination of Herbal and Non-Herbal Components

In some preferred embodiments, the product comprises one or more components selected from the group consisting of peppermint oil and menthol, and one or more components selected from the group consisting of ginger oil, vitamin E, and allantoin. In some highly preferred embodiments, the product comprises peppermint oil, menthol, ginger oil, vitamin E, and allantoin.

In some preferred embodiments, the combined weight percentage of the herbal and non-herbal therapeutic components of the product comprises about 2-30% of the total product weight. In some more preferred embodiments, the combined weight percentage of the herbal and non-herbal therapeutic components of the product comprises about 5-15% of the total product weight. In some highly preferred embodiments, the combined weight percentage of the herbal and non-herbal therapeutic components of the product comprises about 9-11% of the total product weight.

In some preferred embodiments, the product comprises about 2-15% by weight peppermint oil. In some more preferred embodiments, the product comprises about 4-6% by weight peppermint oil. In some highly preferred embodiments, the product comprises about 5% by weight peppermint oil.

In some preferred embodiments, the product comprises about 0.125-16% by weight menthol sourced from menthol crystals or one or more other sources of isolated menthol in addition to any menthol sourced from peppermint oil. In some more preferred embodiments, the product comprises about 0.5-5% by weight menthol sourced from menthol crystals or one or more other sources of isolated menthol in addition to any menthol sourced from peppermint oil. In some highly preferred embodiments, the product comprises about 1.25% by weight menthol sourced from menthol crystals or one or more other sources of isolated menthol in addition to any menthol sourced from peppermint oil.

In some preferred embodiments, the product comprises about 0.05-10% by weight ginger oil. In some more preferred embodiments, the product comprises about 2-4% by weight ginger oil. In some highly preferred embodiments, the product comprises about 3% by weight ginger oil.

In some preferred embodiments, the product comprises about 0.05-3% by weight vitamin E. In some more preferred embodiments, the product comprises about 0.1-0.5% by weight vitamin E. In some highly preferred embodiments, the product comprises about 0.2% by weight vitamin E.

In some preferred embodiments, the product comprises about 0.05-3% by weight allantoin. In some more preferred embodiments, the product comprises about 0.1-0.5% by weight allantoin. In some highly preferred embodiments, the product comprises about 0.2% by weight allantoin.

In some highly preferred embodiments, the product comprises about 5% by weight peppermint oil, about 1.25% by weight menthol sourced from menthol crystals or one or more other sources of isolated menthol in addition to any menthol sourced from peppermint oil, about 3% by weight ginger oil, about 0.2% by weight vitamin E, and about 0.2% by weight allantoin.

### Preparation of Product

In some embodiments, the product is prepared by: (1) melting the wax base, (2) adding peppermint oil, menthol, or peppermint oil and menthol to the liquified wax, wherein the addition is sequential if more than one component is being added to the liquefied wax, (3) sequentially adding other components to the solution, and (4) applying low heat continuously with stirring for approximately 5 minutes, wherein the temperature of the solution does not increase to a temperature which would cause an appreciable amount of vapor to form. The steps may preferably be carried out in order.

In other embodiments, the product is prepared by: (1) adding peppermint oil, menthol, or peppermint oil and menthol to the carrier liquid to yield a solution, wherein the addition is sequential if more than one component is being added to the carrier liquid, (2) sequentially adding other components to the solution, and (3) stirring the solution at ambient temperature. The steps may preferably be carried out in order.

In other embodiments, the product is prepared by: (1) adding peppermint oil, menthol, or peppermint oil and menthol to the carrier liquid to yield a solution, wherein the addition is sequential if more than one component is being added to the carrier liquid, (2) sequentially adding other components to the solution, and (3) applying low heat continuously with stirring for approximately 5 minutes, wherein the temperature of the solution does not increase to a temperature which would cause an appreciable amount of vapor to form. The steps may preferably be carried out in order.

In some preferred embodiments, the product is prepared by: (1) adding water soluble components of the product to water or a solvent comprising water and a polar solvent to yield a water phase, (2) adding non-water soluble components of the product to a non-aqueous solvent component of the carrier liquid to yield an oil phase, and (3) mixing the water phase and the oil phase to yield a product mixture. The steps may preferably be carried out at ambient temperature. In either of or both steps (1) and (2), the product components may be added to the solvent pre-mixed or pre-dissolved in another solvent as appropriate. The product mixture obtained may preferably be homogeneous.

The final formulations may be liquids, creams, lotions, ointments, water-in-oil emulsions, oil-in-water emulsions, semi-solids, transdermal patches, or any other acceptable forms suitable for effective dermal delivery. It is assumed that one skilled in the art could create the appropriate final formulations via known processes or by effectively adapting available techniques and principles to achieve the desired outcome.

### Device and Delivery Mechanisms

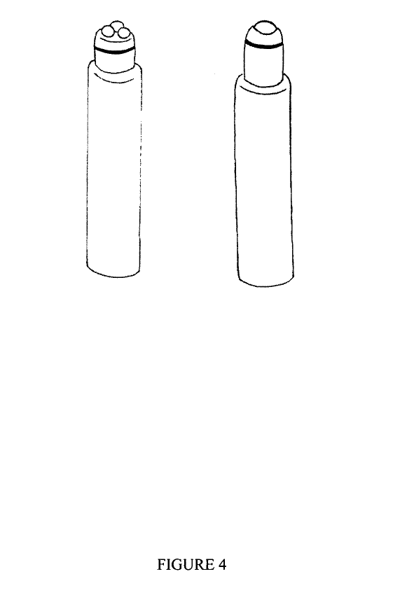

The product may be applied to one or more MTPs of an individual experiencing a headache using a bottle comprising one or more roller balls. In some embodiments, the roller ball(s) comprise stainless steel. The roller ball(s) may project outward from the superior surface of the bottle. The bottle may preferably further comprise a removable cap to protect the roller ball(s) when the bottle is not in use. The roller ball(s) may preferably be sufficiently firm to provide sufficient flow to administer sufficient product to provide the intended quantity of product but also sufficiently flexible, absorbent, or flexible and absorbent such that there will be no dripping. In preferred embodiments, the roller ball(s) may comprise pump-and-click type roller ball(s) that administer a specified dose of product. In some embodiments, the roller ball(s) may produce a massaging effect when used to apply the product to one or more MTPs of an individual experiencing a headache. The bottle may preferably comprise one or three roller balls. FIG. 4 illustrates bottles with one and three roller balls that may be used to deliver the product.

The product may also be applied to one or more MTPs of an individual experiencing a headache using an applicator stick. The applicator stick may comprise a wax-based applicator stick comprising the product that is pushed outward beyond the superior edge of an applicator stick housing using a twisting disc at the bottom of the applicator stick housing. The applicator stick housing comprises the applicator stick and may preferably further comprise a cap to protect the applicator stick when the latter is not in use. The design and product delivery mechanism of the applicator stick may be analogous to the product delivery mechanism of commercially available applicator sticks for lip balms, although the applicator stick and applicator stick housing may preferably be larger than typical commercially available applicator sticks for lip balms.

The product may be provided in a container such that it may be removed by hand and applied by hand to one or more MTPs of an individual experiencing a headache. The container may be analogous in design and shape to commercially available disc-shaped containers containing lip balms or other creams. The container may preferably further comprise a cap to protect the contents of the container when not in use.

It is assumed that one skilled in the art may devise other appropriate delivery mechanisms for the product to achieve the desired outcome.

### Methods of Applying Product

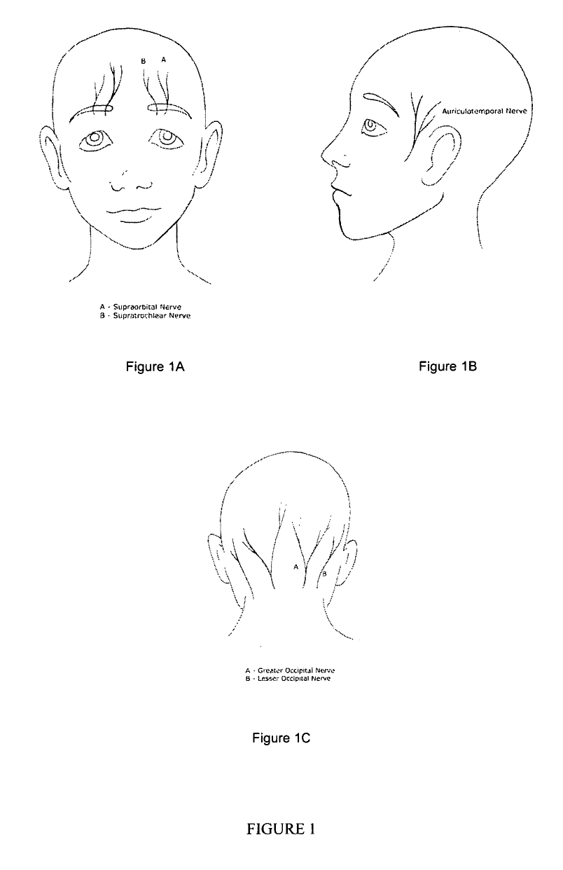

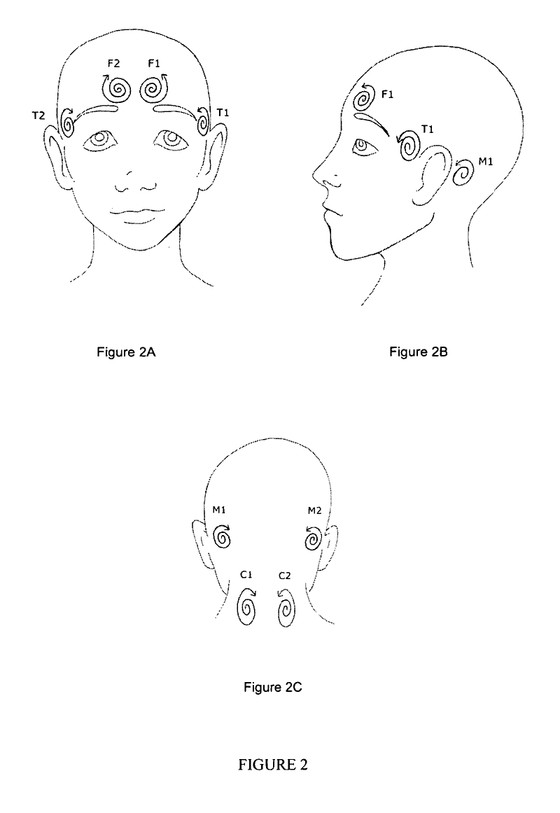

In some embodiments, the product may be applied to one or more MTPs of an individual experiencing a headache by rubbing in a circular, massaging motion. The product may preferably be applied to the temporal, frontal, mastoid, or cervical MTPs, or a combination thereof,⁵⁰ as shown in FIG. 2. Application to the temporal MTPs may deliver the product to the auriculotemporal distribution. Application to the frontal MTPs may deliver the product to the supraorbital and supratrochlear distribution. Application to the mastoid MTPs may deliver the product to the lesser occipital and greater auricular distribution. Application to the cervical MTPs may deliver the product to the cervical nerve roots. FIG. 1 illustrates these targeted nerve distributions. ⁵⁰See Levin, M. “Nerve Blocks in the Treatment of Headache.” Neurotherapeutics 2010, 7(2):197.

### Combination Therapy

The disclosed product may be used to treat common headache syndromes in combination with other co-morbidities including fibromyalgia, arthritis, and premenstrual syndrome.

### Ancillary Beneficial Effects

In some preferred embodiments, use of the product may cause the individual using the product to feel energized, refreshed, and more alert, and to experience a sense of well-being.

### Prophylaxis

In some preferred embodiments, the product may be used prophylactically to prevent headaches.

The examples above are intended as illustrative and are not intended to limit or otherwise restrict the invention. All references cited herein are expressly incorporated by reference.

What is claimed is:

1. A method of treating headaches wherein a product for headache relief comprising one or more herbal therapeutic agents selected from the group consisting of herbal or other non-herbal natural products listed in one or more of the over-the-counter (OTC) nonprescription drug product monographs established by the United States Food and Drug Administration (FDA) and other herbal or other non-herbal natural products not monographed by the FDA that have one or more therapeutic properties, and a pharmaceutically acceptable carrier base, is applied to one or more myofascial trigger points of an individual experiencing a headache.

2. The method of claim 1 wherein the product comprises one or more herbal therapeutic agents selected from the group consisting of peppermint oil, menthol, ginger oil, vitamin E, green tea extract, catechin, Korean ginseng, American ginseng, caffeine, β-endorphin receptor agonists, valerian, passionflower, clary sage, rosemary, black seed oil, camphor, clove oil, capsaicin, methyl salicylate, garlic oil, lemon oil, tea tree oil, castor oil, eucalyptus oil, jojoba oil, flaxseed oil, lanolin, cumin oil, and tansy oil.

3. The method of claim 2 wherein the herbal therapeutic agents comprise one or more herbal therapeutic agents selected from the group consisting of peppermint oil, menthol, ginger oil, vitamin E, green tea extract, catechin, Korean ginseng, American ginseng, caffeine, β-endorphin receptor agonists, valerian, passionflower, clary sage, rosemary, and black seed oil.

4. The method of claim 3 wherein the herbal therapeutic agents comprise one or more herbal therapeutic agents selected from the group consisting of peppermint oil, menthol, ginger oil, and vitamin E.

5. The method of claim 1 wherein the product further comprises non-herbal therapeutic agents comprising non-steroidal anti-inflammatory drugs (NSAIDs), steroidal anti-inflammatory agents, analgesics, or anesthetics, or any combinations thereof.

6. A product for headache relief comprising one or more components selected from the group consisting of peppermint oil and menthol, one or more components selected from the group consisting of vitamin E and allantoin, and a pharmaceutically acceptable carrier base.

7. The product of claim 6 wherein the product further comprises ginger oil.

8. The product of claim 6 wherein the product further comprises non-herbal therapeutic agents such as non-steroidal anti-inflammatory drugs (NSAIDs), steroidal anti-inflammatory agents, analgesics, or anesthetics, or any combinations thereof.

9. The product of claim 7 wherein the product further comprises allantoin.

10. The product of claim 9 wherein the combined weight percentage of the herbal and non-herbal therapeutic agents that comprise the product is about 2-30% of the total product weight.

11. The product of claim 9 wherein the combined weight percentage of the herbal and non-herbal therapeutic agents that comprise the product is about 5-15% of the total product weight.

12. The product of claim 9 wherein the combined weight percentage of the herbal and non-herbal therapeutic agents that comprise the product is about 9-11% of the total product weight.

13. The product of claim 9 wherein the product comprises about 2-15% by weight peppermint oil, about 0.125-16% by weight menthol sourced from menthol crystals or one or more other sources of isolated menthol in addition to any menthol sourced from peppermint oil, about 0.05-10% by weight ginger oil, about 0.05-3% by weight vitamin E, and about 0.05-3% by weight allantoin.

14. The product of claim 9 wherein the product comprises about 4-6% by weight peppermint oil, about 0.5-5% by weight menthol sourced from menthol crystals or one or more other sources of isolated menthol in addition to any menthol sourced from peppermint oil, about 2-4% by weight ginger oil, about 0.1-0.5% by weight vitamin E, and about 0.1-0.5% by weight allantoin.

15. The product of claim 12 wherein the product comprises about 5% by weight peppermint oil, about 1.25% by weight menthol sourced from menthol crystals or one or more other sources of isolated menthol in addition to any menthol sourced from peppermint oil, about 3% by weight ginger oil, about 0.2% by weight vitamin E, and about 0.2% by weight allantoin.

16. The product of claim 9 wherein the pharmaceutically acceptable carrier base comprises one or more dermal penetration enhancers.

17. A method of treating headaches wherein the product of claim 16 is applied to one or more myofascial trigger points of an individual experiencing a headache.

18. The method of claim 17 wherein the product is applied to one or more myofascial trigger points of an individual experiencing a headache using a bottle comprising one or more roller balls.

19. The method of claim 18 wherein the one or more roller balls are sufficiently firm to provide sufficient flow to administer sufficient product to provide the intended quantity of product, but are also sufficiently flexible, absorbent, or flexible and absorbent such that there is no dripping.

20. The method of claim 19 wherein the one or more roller balls produce a massaging effect when used to apply the product to one or more myofascial trigger points of an individual experiencing a headache.

21. The method of claim 17 wherein the product is applied to one or more myofascial trigger points of an individual experiencing a headache by rubbing in a circular motion.

22. The method of claim 17 wherein the product is used prophylactically to prevent headaches.

23. The method of claim 17 wherein the product is used to treat common headache syndromes in combination with other co-morbidities.

24. The method of claim 2 wherein use of the product causes the individual to feel energized and refreshed.

25. A method of providing an individual with feelings of alertness, increased energy, and well-being wherein a product comprising one or more herbal therapeutic agents selected from the group consisting of peppermint oil, menthol, ginger oil, vitamin E, green tea extract, catechin, Korean ginseng, American ginseng, caffeine, β-endorphin receptor agonists, valerian, passionflower, clary sage, rosemary, black seed oil, camphor, clove oil, capsaicin, methyl salicylate, garlic oil, lemon oil, tea tree oil, castor oil, eucalyptus oil, jojoba oil, flaxseed oil, lanolin, cumin oil, and tansy oil, and a pharmaceutically acceptable carrier base, is applied to one or more myofascial trigger points of the individual.
